(19) United States
(12) Patent Application Publication
(10) Pub. No.: US 2003/0175717 A1
(43) Pub. Date: Sep. 18, 2003

(54) Apparatus and method for predicting treatment response of cancer

(76) Inventors: Shulin Li, Troy, MI (US); Ehah Y.N. Hanna, Little Rock, AR (US); James Buen, Little Rock, AR (US)

Correspondence Address: J.M. (Mark) Gilbreth, Gilbreth & Associates, P.C., P.O. Box 2428, Bellaire, TX, 77402-2428, US

(21) Appl. No.: 10/097,525

(22) Filed: Mar. 14, 2002

(51) Int. Cl. C12Q 1/68; G06F 19/00; G01N 33/48; G01N 33/50; C12M 1/34

(52) U.S. Cl. 435/6; 702/20; 435/287.2

(57) ABSTRACT

The present invention is directed to the identification of prognostic molecular markers of cancer and methods of predicting treatment response of a cancer. The invention further discloses molecular chips useful in predicting whether a cancer will respond to treatment. Preferably, the treatment is radiation therapy. Methods of using and methods of making the molecular chips of the invention are also disclosed.

### BACKGROUND OF THE INVENTION

1. Field of the Invention

The present invention relates to cancer and the treatment of cancer. In another aspect, the present invention relates to apparatus and methods for predicting treatment response of a cancer. In even another aspect, the present invention relates to molecular chips and methods of using such chips for predicting whether a cancer will be radiation-sensitive or radiation-resistant. In still another aspect, the present invention relates to molecular chips and methods of using said chips for predicting whether a cancer will respond to chemotherapy. In yet another aspect, the present invention relates to molecular chips and methods of using said chips for predicting whether a cancer will respond to concomitant chemoradiotherapy. In even still another aspect, the present invention relates to molecular chips and methods of using such chips for predicting whether a squamous cell carcinoma of the head and neck will respond to radiation therapy.

2. Description of the Related Art

If detected in early stages, many types of cancer can be effectively treated using treatments comprising surgery, radiation therapy, chemotherapy, and/or any combination thereof. For example, if diagnosed in its early stages, squamous cell carcinoma (SCC) of the upper aerodigestive tract has excellent control rates with either surgery or radiation. However, when detected in an advanced stage, SCC of the head and neck (SCCHN) has a significantly high death rate with reported 5-year survival rates of 38-60%, despite the development of aggressive and multi-modal treatments (Kramer, S., Gelber, R. D., Snow, J. B., Marcial, V. A., Lowry, L. D., Davis, L. W., and Chandler, R. Combined radiation therapy and surgery in the management of advanced head and neck cancer: final report of study 73-03 of the Radiation Therapy Oncology Group. Head Neck Surg., 10: 19-30, 1987.; Lavertu, P., Adelstein, D. J., Saxton, J. P., Secic, M., Eliachar, I., Strome, M., Larto, M, A., and Wood, B. G. Aggressive concurrent chemoradiotherapy for squamous cell head and neck cancer: an 8-year single-institution experience. Arch Otolaryngol Head Neck Surg., 125: 142-148, 1999; Wanebo, H. J., Glicksman, A. S., Landman, C., Slotman, G., Doolittle, C., Clark, J., and Koness, R. J. Preoperative cisplatin and accelerated hyperfractionated radiation induces high tumor response and control rates in patients with advanced head and neck cancer. Am J Surg., 170: 512-516, 1995; Dragovic, J., Doyle, T. J., Tilchen, E. J., Nichols, R. D., Benninger, M. S., Carlson, E. R., Boyd, S. B., and Jacobsen, G. R. Accelerated fractionation radiotherapy and concomitant chemotherapy in patients with stage 1V inoperable head and neck cancer. Cancer, 76: 1655-1661, 1995.).

Recently, alternative therapy protocols utilizing concomitant chemoradiotherapy have appeared in the art in hope that the morbidity associated with advanced stage SCCHN can be avoided or decreased. Although the success rate of chemoradiotherapy is generally high, there are patients whose cancers fail to respond to this treatment. Thus, after having gone through treatment for which their cancer is relatively resistant, these patients must usually then undergo a difficult surgery. Clearly, the ability to accurately predict treatment response of a cancer would be a valuable, time-saving tool for both the patient and the physician, and would minimize patient suffering and optimize time and treatment strategy.

To date, most of the studies in the art aimed at identifying molecular markers for prediction of radiation response are based on the observation of gene expression using immunostaining, Northern blot, or Western blot analysis of a single or several genes. Because the results vary among different studies and, as discussed below, are often times contradictory, predicting treatment response of a cancer based on a single gene is inaccurate.

Results from in vitro cell culture study and in vivo preclinical and clinical studies suggest the expression of certain oncogenes and/or inactivation of tumor suppressor genes, as well as apoptosis and cell cycle genes, may alter the cellular radiation. In general, in vitro transfection of transforming oncogenes such as, for example, v-myc (FitzGerald, T. J., Santucci, M. A., Das, I., Kase, K., Pierce, J. H., and Greenberger, J. S. The v-abl, c-fms, or v-myc oncogene induces gamma radiation resistance of hematopoietic progenitor cell line 32d cl 3 at clinical low dose rate. Int. J. Radiat. Oncol. Biol. Phys., 21:1203-1210, 1991; Iliakis, G., Metzger, L., Muschel, R. J., and McKenna, W. G. Induction and repair of DNA double strand breaks in radiation-resistant cells obtained by transformation of primary rat embryo cells with the oncogenes H-ras and v-myc. Cancer Res., 50: 6575-6579, 1990); Ha-ras (Iliakis, G., Metzger, L., Muschel, R. J., and McKenna, W. G. Induction and repair of DNA double strand breaks in radiation-resistant cells obtained by transformation of primary rat embryo cells with the oncogenes H-ras and v-myc. Cancer Res., 50: 6575-6579, 1990; McKenna, W. G., Weiss, M. C., Endlich, B., Ling, C. C., Bakanauskas, V. J., Kelsten, M. L., and Muschel, R. J. Synergistic effect of the v-myc oncogene with H-ras on radioresistance. Cancer Res., 50: 97-102, 1990; Miller, A. C., Kariko, K., Myers, C. E., Clark, E. P., and Samid, D. Increased radioresistance of ras-transformed human osteosarcoma cells and its modulation by lovastatin, an inhibitor of p21ras isoprenylation. Int. J. Cancer, 53: 302-307, 1993; Su, L. N., and Little, J. B. Prolonged cell cycle delay in radioresistant human cell lines transfected with activated ras oncogene and/or simian virus 40 T-antigen. Radiat. Res., 133: 73-79, 1993.); Ki and N-ras (Chang, E. H., Pirollo, K. F., Zou, Z. Q., Cheung, H. Y., Lawler, E. L., Garner, R., White, E., Bernstein, W. B., Fraumeni, J. W., Jr., and Blattner, W. A. Oncogenes in radioresistant, noncancerous skin fibroblasts from a cancer-prone family. Science, 237: 1036-1039, 1987; Sklar, M. D. The ras oncogenes increase the intrinsic resistance of NIH 3T3 cells to ionizing radiation. Science, 239: 645-647, 1988; FitzGerald, T. J., Henault, S., Sakakeeny, M., Santucci, M. A., Pierce, J. H., Anklesaria, P., Kase, K., and Das, I. Expression of transfected recombinant oncogenes increases radiation resistance of clonal hematopoietic and fibroblast cell lines selectively at clinical low dose rate. Radiat. Res., 122: 44-52, 1990; Samid, D., Miller, A. C., Rimoldi, D., Gafner, J., and Clark, E. P. Increased radiation resistance in transformed and nontransformed cells with elevated ras proto-oncogene expression. Radiat. Res., 126: 244-250, 1991); rat (Miller, A. C., Kariko, K., Myers, C. E., Clark, E. P., and Samid, D. Increased radioresistance of ras-transformed human osteosarcoma cells and its modulation by lovastatin, an inhibitor of p21ras isoprenylation. Int. J. Cancer, 53: 302-307, 1993; Kasid, U. Pfeifer, A., Brennan, T., Beckett, M., Weichselbaum, R. R., Dritschilo, A., and Mark, G. E. Effect of antisense c-raf-1 on tumorigenicity and radiation sensitivity of a human squamous carcinoma. Science, 243: 1354-1356, 1989; Kasid, U., Pfeifer, A., Weichselbaum, R. R., Dritschilo, A., and Mark G. E. The rat oncogene is associated with a radiation-resistant human laryngeal cancer. Science, 237: 1039-1041, 1987); v-abl (FitzGerald, T. J., Santucci, M. A., Das, I., Kase, K., Pierce, J. H., and Greenberger, J. S. The v-abl, c-fms, or v-myc oncogene induces gamma radiation resistance of hematopoietic progenitor cell line 32d cl 3 at clinical low dose rate. Int. J. Radiat. Oncol. Biol. Phys., 21:1203-1210, 1991; Chiang, C. S., Sawyers, C. L., and McBridge, W. H. Oncogene expression and cellular radiation resistance: a modular role for c-myc. Mol. Diagn., 3: 21-28, 1998); bcr-abl (Chiang, C. S., Sawyers, C. L., and McBridge, W. H. Oncogene expression and cellular radiation resistance: a modular role for c-myc. Mol. Diagn., 3: 21-28, 1998); v-mos (Chiang, C. S., Sawyers, C. L., and McBridge, W. H. Oncogene expression and cellular radiation resistance: a modular role for c-myc. Mol. Diagn., 3: 21-28, 1998; Suzuki, K., Watanabe, M., and Miyoshi, J. Differences in effects of oncogenes on resistance of gamma rays, ultraviolet light, and heat shock. Radiat Res. 129: 157-162, 1992); v-fes (FitzGerald, T. J., Santucci, M. A., Das, I., Kase, K., Pierce, J. H., and Greenberger, J. S. The v-abl, c-fms, or v-myc oncogene induces gamma radiation resistance of hematopoietic progenitor cell line 32d cl 3 at clinical low dose rate. Int. J. Radiat. Oncol. Biol. Phys., 21:1203-1210, 1991); c-cot (Suzuki, K., Watanabe, M., and Miyoshi, J. Differences in effects of oncogenes on resistance of gamma rays, ultraviolet light, and heat shock. Radiat Res. 129: 157-162, 1992); v-src (Shimm, D. S., Miller, P. R., Lin, T., Moulinier, P. P. and Hill, A. B. Effects of v-src oncogene activation on radiation sensitivity in drug-sensitive and in multidrug-resistant rat fibroblasts. Radiat. Res., 129: 149-156, 1992); and simian virus 40 T antigen (Su, L. N., and Little, J. B. Prolonged cell cycle delay in radioresistant human cell lines transfected with activated ras oncogene and/or simian virus 40 T-antigen. Radiat. Res., 133: 73-79, 1993), increases cellular radioresistance. Extended research efforts demonstrate that the inhibition of raf expression by antisense RNA is sufficient for restoring radiosensitivity in human SCC. Increased expression of endogenous oncogenes has also been correlated with radiation resistance in tumor cell lines and tissues. For example, high levels of EGFR, c-myc and Ki67 expression increased radiation resistance of SCCHN (Miyaguchi, M., Takeuchi, T., Morimoto, K., and Kubo, T. Correlation of epidermal growth factor receptor and 5 radiosensitivity in human maxillary cell lines. Acta Otolarygol., 118: 428-431, 1998), increased c-raf expression was implicated in the radioresistance of SCCHN (Riva, C., Lavieille, J. P., Reyt, E., Brambilla, E., Lunardi, J., and Brambilla, C. Differential c-myc, c-jun, c-raf and p53 expression in squamous cell carcinoma of the head and neck: implication in drug and radioresistance. Eur. J. Cancer, 311B: 384-391, 1995), and overexpression of c-jun and c-H-ras correlate to radioresistance of clinical laryngeal tumor samples (Miura, K., Suzuki, S., Tanita, is J., Shinkawa, H., Satoh, K., and Tsuchida, S. Correlated expression of glutathione S-transferase-pi and c-Jun or other oncogene products in human squamous cell carcinomas of the head and neck: relevance to relapse after radiation therapy. Jpn. J. Cancer Res., 88: 143-151, 1997). Unfortunately, results from radioresistance studies which present contradictions have also been reported in the art.

For example, bcr-abl has been reported to induce radiosensitivity in hematopoietic cells (Shimm, D. S., Miller, P. R., Lin, T., Moulinier, P. P. and Hill, A. B. Effects of v-src oncogene activation on radiation sensitivity in drug-sensitive and in multidrug-resistant rat fibroblasts. Radiat. Res., 129: 149-156, 1992), as did c-myc and Ha-ras in melanoma cells and Rat-1 cells after low radiation doses (2-4 Gy) (Pomp, J., Ouwerkerk, I. J., Hermans, J., Wondergem, J., Cornelisse, C. J., Leer, J. W., and Schrier, P. I. The influence of the oncogenes NRAS and MYC on the radiation sensitivity of cells of a human melanoma cell line. Radiat. Res., 146: 374-381, 1996; Garden, A. S., Meyn, R. E., Weil, M. M., Lebovitz, R. M., and Lieberman, M. W. The influence of ras oncogene expression on radiation response in the Rat-1 cell. Int. J. Radiat. Biol., 62: 307-311, 1992). Conflicting results have also resulted from studies with c-raf-1, which implicate a correlation between high levels of c-raf-1 expression and radiosensitivity of human cell lines, rather than radioresistance (Warenius, H. M., Browning, P. G., Britten, R. A., Peacock, J. A., and Rapp, U. R. C-raf-1 proto-oncogene expression relates to radiosensitivity rather than radioresistance. Eur. J. Cancer, 30A: 369-375, 1994). In addition, radiation-sensitive and radiation-resistant ras-transformed clones have been obtained from a single transfection experiment (Harris, J. F., Chambers, A. F., and Tam, A. S. Some ras-transformed cells have increased radiosensitivity and decreased repair of sublethal radiation damage. Somat. Cell Mol. Genet., 16: 39-48, 1990). Thus, colonial diversity within cancer cells makes it difficult to predict a correlation between oncogene expression and radiation response (Ling, C. C., and Endlich, B. Radioresistance induced by oncogenic transformation. Radiat. Res., 120: 267-279, 1989).

Adding to the complexity is the fact that the ratio and combination of oncogenes may implicate the prognostic function of the oncogenes. Higher ratios of c-myc and c-jun expression have been implicated in conferring radiation-sensitivity, with lower ratios possibly being predictive of chemoresistance (Riva, C., Lavieille, J. P., Reyt, E., Brambilla, E., Lunardi, J., and Brambilla, C. Differential c-myc, c-jun, c-raf and p53 expression in squamous cell carcinoma of the head and neck: implication in drug and radioresistance. Eur. J. Cancer, 31B: 384-391, 1995). The combination of ras and c-myc have been reported to function synergistically in the radiation-resistance effect in rat embryo lung cells (McKenna, W. G., Weiss, M. C., Endlich, B., Ling, C. C., Bakanauskas, V. J., Kelsten, M. L., and Muschel, R. J. Synergistic effect of the v-myc oncogene with H-ras on radioresistance. Cancer Res., 50: 97-102, 1990; Sklar, M. D., Thompson, E., Welsh, M. J., Liebert, M., Harney, J., Grossman, H. B., Smith, M., and Prochownik, E. V. Depletion of c-myc with specific antisense sequences reverses the transformed phenotype in ras oncogene-transformed NIH 3T3 cells. Mol. Cell. Biol., 11: 3699-3710, 1991; Kyprianou, N., King, E. D., Bradbury, D., and Rhee, J. G. bcl-2 over-expression delays radiation-induced apoptosis without affecting the clonogenic survival of human prostate cancer cells. Int. J. Cancer, 70: 341-348, 1997), whereas the combination of c-myc and v-abl abolishes the radioresistance, and the transfection of either gene alone results in radioresistance.

Apoptosis is considered a major molecular mechanism of radiation-induced cell killing of various tumor cells. Overexpression of apoptotic suppressing genes or downregulation of pro-apoptotic genes can potentially render tumor cells radioresistant (Weller, M., Malipiero, U., Aguzzi, A., Reed, J. C., and Fontana, A. Protooncogene bcl-2 gene transfer abrogates Fas/APO-1 antibody-mediated apoptosis of human malignant glioma cells and confers resistance to chemotherapeutic drugs and therapeutic irradiation. J. Clin. Invest., 95: 2633-2643, 1995). The most widely studied genes for radiation response are those of the bcl-2 family, and the predictive value of bcl-2 for radiation resistance has been examined in breast cancer, prostate cancer, and head and neck cancer (Gasparini, G., Barbareschi, M., Doglioni, C., Palma, P. D., Mauri, F. A., Boracchi, P., Bevilacqua, P., Caffo, O., Morelli, L., and Verderio, P., Pezzella, F., and Harris, A. C. Expression of bcl-2 protein predicts efficacy of adjuvant treatments in operable node-positive breast cancer. Clin. Cancer Res., 1: 189-198, 1995; Gallo, O., Boddi, V., Calzolari, A., Simonetti, L., Trovati, M., and Bianchi, S. bcl-2 protein expression correlates with recurrence and survival in early stage head and neck cancer treated by radiotherapy. Clin. Cancer Res., 2: 261-267, 1996; Hague, A., Moorghen, M., Hicks, D., Chapman, M., Paraskeva, C. BCL-2 expression in human colorectal adenomas and carcinomas. Oncogene, 9: 3367-3370, 1994). While overexpression of bcl-2 increases radiation resistance in multiple tumor cells (Weller, M., Malipiero, U., Aguzzi, A., Reed, J. C., and Fontana, A. Protooncogene bcl-2 gene transfer abrogates Fas/APO-1 antibody-mediated apoptosis of human malignant glioma cells and confers resistance to chemotherapeutic drugs and therapeutic irradiation. J. Clin. Invest., 95: 2633-2643, 1995; Epperly M W, Santucci M A, Reed J C, et al. Expression of the human bcl-2 transgene increases the radiation resistance of a hemotopoietic cell line. Radiat. Oncol. Invest., 2: 77-83, 1994; Mackey, T. J., Borkowski, A., Amin, P., Jacobs, S. C., and Kyprianou, N. bcl-2/bax ratio as a predictive marker for therapeutic response to radiotherapy in patients with prostate cancer. Urology, 52: 1085-1090, 1998; Gasparini, G., Barbareschi, M., Doglioni, C., Palma, P. D., Mauri, F. A., Boracchi, P., Bevilacqua, P., Caffo, O., Morelli, L., and Verderio, P., Pezzella, F., and Harris, A. C. Expression of bcl-2 protein predicts efficacy of adjuvant treatments in operable node-positive breast cancer. Clin. Cancer Res., 1: 189-198, 1995), this bcl-2 function is antagonized by bax, another bcl-2 family member.

Contradictory results regarding bcl-2 overexpression and radiation response have been reported in studies with bladder cancer and colorectal adenocarcinoma (Kaklamanis, L., Savage, A., Whitehouse, R., Doussis-Anagnostopoulou, I., Biddolph, S., Tsiotos, P., Mortensen, N., Gatter, K. C., and Harris, A. L. Bcl-2 protein expression: association with p53 and prognosis in colorectal cancer. Br. J. Cancer, 77: 1864-1869, 1998; Manne, U., Myers, R. B., Moron, C., Poczatek, R. B., Dillard, S., Weiss, H., Brown, D., Srivastava, S., And Grizzle, W. E. Prognostic significance of Bcl-2 expression and p53 nuclear accumulation in colorectal adenocarcinoma. Int. J. Cancer, 74: 346-358, 1997; Watson, A. J., Merritt, A. J., Jones, L. S., Askew, J. N., Anderson, E., Becciolini, A., Balzi, M., Potten, C. S., and Hickman, J. A. Evidence of reciprocity of bcl-2 and p53 expression in human colorectal adenomas and carcinomas. Br. J. Cancer, 73: 889-895, 1996; Ofner, D., Riehemann, K., Maier, H., Riedmann, B., Nehoda, H., Totsch, M., Bocker, W., Jasani, B., and Schmid, K. W. Immunohistochemically detectable bcl-2 expression in colorectal carcinoma: correlation with tumour stage and patient survival. Br. J. Cancer, 72: 981-985, 1995; Pollack, A., Wu, C. S., Czerniak, B., Zagars, G. K., Benedict, W. F., and McDonnell, T. J. Abnormal bcl-2 and pRb expression are independent correlates of radiation response in muscle-invasive bladder cancer. Clin. Cancer Res., 3: 1823-1829, 1997), in which high levels of bcl-2 staining correlated with radiation sensitivity (Fu, C. G., Tominaga, O., Nagawa, H., Nita, M. E., Masaki, T., Ishimaru, G., Higuchi, Y., Tsuruo, T., and Muto, T. Role of p53 and p21/WAF1 detection in patient selection for preoperative radiotherapy in rectal cancer patients. Dis. Colon Rectum, 41: 68-74, 1998). Similar results were observed in SCCHN by Western blot analysis (unpublished). Finally, there are results that suggest a lack of correlation between bcl-2 expression and tumor radiosensitivity (Scott, N., Hale, A., Deakin, M., Hand, P. Adab, F. A., Hall, C., Williams, G. T., and Elder, J. B. A histopathological assessment of the response of rectal adenocarcinoma to combination chemo-radiotherapy: relationship to apoptotic activity, p53 and bcl-2 expression. Eur. J. Surg. Oncol., 24: 169-173, 1998; Tannapfel, A., Nusslein, S., Fietkau, R., Katalinic, A., Kockerling, F., and Wittekind, C. Apoptosis, proliferation, bax, bcl-2 and p53 status prior to and after preoperative radiochemotherapy for locally advanced rectal cancer. Int. J. Radiat. Oncol. Biol. Phys., 41: 585-591, 1998; Stammler, G., Pommerenke, E. W., Mattern, J., and Volm, M. Effects of single doses of irradiation on the expression of resistance-related proteins in murine NIH 3T3 and human lung carcinoma cells. Carcinogenesis, 16: 2051-2055, 1995).

Thus, in spite of advancements in the art, molecular markers useful for accurately predicting treatment response of a cancer are not well validated and the methods used for comparison are greatly varied, which in turn influences the accuracy and consistency of results. In addition, radiation-resistance is determined by multiple cellular events controlled by a large pool of genes and their interactions, and it is critical to compare the expression of these markers simultaneously. Thus, there remains a need in the art for apparatus and methods useful in accurately predicting the treatment response of a cancer.

There is another need in the art for molecular chips useful in predicting treatment response of a cancer.

There is even another need in the art for methods of using said molecular chips to predict whether a cancer will respond to radiation therapy, chemotherapy, and/or a combination thereof.

There is still another need in the art for methods of making such prognostic chips.

There is yet another need in the art for molecular chips and methods of using said chips to predict whether a squamous cell carcinoma will respond to radiation therapy, chemotherapy, and/or a combination thereof.

These and other needs will become apparent to those of skill in the art upon review of this specification, including its drawings, claims and appendix.

### SUMMARY OF THE INVENTION

It is an object of the present invention to provide apparatus and methods useful in accurately predicting the treatment response of a cancer.

It is another object of the present invention to provide molecular chips useful in predicting treatment response of a cancer.

It is even another object of the present invention to provide methods of using said molecular chips to predict whether a cancer will respond to radiation therapy, chemotherapy, and/or a combination thereof.

It is still another object of the present invention to provide methods for making such prognostic chips.

It is yet another object of the present invention to provide molecular chips and methods of using said chips to predict whether a squamous cell carcinoma will respond to radiation therapy, chemotherapy, and/or a combination thereof.

According to one embodiment of the present invention there is provided a molecular chip useful for predicting treatment response of a cancer. Generally the prognostic molecular chip comprises an array of nucleic acid sequences, such as, for example, cDNA sequences, which correspond to a large group of molecular marker genes. The molecular markers may be any group of genes, so long as the genes exhibit a differential expression pattern in treatment-sensitive and treatment-resistant samples.

According to another embodiment of the invention there is provided a method for predicting treatment response of a cancer in a patient. The method generally comprises the step of contacting a portion of a sample from a cancer of a patient with a molecular chip of the invention. Preferably the sample is from a biopsy.

These and other embodiments of the present invention will become apparent to those of skill in the art upon review of this specification, including its drawings, appendix, and claims.

### BRIEF DESCRIPTION OF THE DRAWINGS

FIG. 1 provides gene expression profile results from a cDNA array hybridization utilizing radioactively-labeled cDNA probes reverse-transcribed from either radiation-resistant or radiation-sensitive cells.

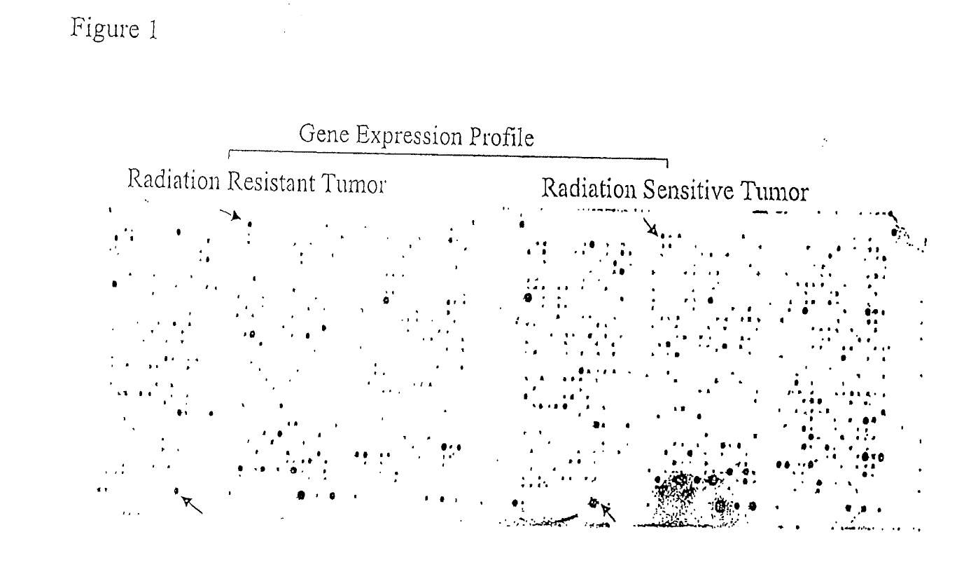

FIG. 2 depicts the cluster analysis of 60 genes differentially expressed in radiation-sensitive (S) and radiation-resistant (R) tissues.

FIG. 3 depicts the radiation response by cluster analysis of two test samples.

### DETAILED DESCRIPTION OF THE INVENTION

The present invention is directed to apparatus useful in predicting treatment response of a cancer, and to methods of using said apparatus to accurately predict whether a cancer will be responsive to a selected treatment. The term “treatment” as used herein includes any and all cancer treatments known in the art, such as, for example, surgery, radiation therapy, chemotherapy, and any combinations thereof. In a preferred embodiment, the treatment is radiation therapy. The present invention comprises the use of a large group of genes that exhibit differential expression patterns in treatment-resistant and treatment-sensitive samples. The group of genes are useful as molecular markers in order to provide a more accurate and reliable prediction of the outcome of a treatment response for a cancer.

According to one embodiment of the invention there is provided a molecular chip. The prognostic molecular chip of the invention is useful for assaying a sample from a patient's cancer in order to predict the treatment response of the cancer. For example, a radiation-response molecular chip of the invention is useful in assaying a sample from a patient's cancer in order to predict whether the cancer is radiation-sensitive or radiation-resistant. Other chips of the invention are useful for predicting response of a cancer to treatments such as, chemotherapy, or chemoradiotherapy. In a preferred embodiment of the invention, the molecular chip is a radiation-response molecular chip. Thus, the chips of the invention allow for assaying a sample in order to predict which treatment method may be most beneficial and effective. Molecular chips may also be referred to as biochips, chips, microchips, and macrochips.

Generally the molecular chips, of the invention comprise an array of nucleic acid sequences, such as cDNA, specific for molecular marker genes of the invention. Thus, a biochip of the invention comprises sequences specific to a large group of molecular marker genes. The genes selected herein for use as molecular marker genes may be any genes that are found to be differentially expressed in treatment-resistant and treatment-sensitive samples. However, for a gene to be selected herein as a molecular marker, the difference in the gene's treatment-resistant expression intensity and the gene's treatment-sensitive expression intensity must meet at least the following criteria: 1) the ratio of treatment-resistant gene expression and treatment-sensitive gene expression is greater than about 3-fold; and 2) the absolute difference between treatment-resistant expression and treatment-sensitive expression is greater than about 75, if the ratio cannot be defined.

In order to determine the expression intensities of genes in treatment-resistant and treatment-sensitive samples, any technique known in the art for efficiently analyzing expression patterns of a large group of genes may be used herein. In a particularly preferred embodiment, a technology known in the art as cDNA array analysis is used to simultaneously analyze the expression intensity patterns of a large number of genes in a treatment-resistant sample and in a treatment-sensitive sample. Those genes whose expression intensities meet the criteria discussed above may then be selected as molecular marker genes of the present invention.

The expression intensity data of the genes selected as molecular markers of the present invention may be subjected to cluster analysis. The raw observation data (the adjusted intensity) is transformed using the equation y=log 10(x+1), where x equals the adjusted intensity from the array analysis, and y equals the transformed adjusted-intensity. The cluster analysis may be performed by any method known in the art, preferably by using weighted pair-group average and Euclidean distance. Software such as STATISTICA (StatSoft, Tulsa, Okla.) is but one example of a commercially available tool for performing cluster analysis. Any such tool known in the art is applicable herein.

The cDNA array assay used herein may be any such array assay known in the art. Numerous arrays are commercially available and known in the art, and all are applicable herein. Preferably the array is a cancer-related gene array. The cDNA array assay is preferably performed according to the manufacturer's instructions.

Numerous types of biochip substrates, also referred to as microarray platforms, such as, for example, nylon and glass, are known in the art, and any such substrate may be used for the chips of the present invention. Apparatus and techniques for adhering nucleic acid sequences to biochips and for analyzing biochips are also known in the art such as for example, photolithography, pipette, drop-touch, piezoelectric (ink-jet), electric, and any such apparatus and technique is applicable for the chips disclosed herein.

According to another embodiment of the invention there is provided a method for predicting treatment response of a cancer in a patient. The method comprises the step of contacting a portion of a test sample from a cancer of a patient with a molecular chip of the invention to obtain an expression intensity pattern of the sample. Preferably the sample is from a biopsy.

In an additional step of the method, the data of the expression intensity pattern obtained from the patient's sample is then transformed by use of the equation y=log 10 (x+1), wherein x equals the expression intensity and y equals the transformed expression intensity. The mathematical method used herein for evaluating overall gene expression of a large group of genes, as opposed to evaluating expression of a single gene or a few genes, greatly reduces the risk of single gene-induced bias.

The method of the invention further comprises the step of performing cluster analysis on the transformed expression intensity of the patient's sample. Generally the cluster analysis comprises weighted pair-group average and Euclidian distance. Software such as, STATISTICA (StatSoft, Tulsa, Okla.) may be used to perform the cluster analysis. A tree diagram may also be generated by the software. The outcome of the cluster analysis my then be compared to cluster analysis results performed on a treatment-resistant control sample and a treatment-sensitive control sample, thus enabling a prediction of whether the cancer from which the test sample was obtained will be resistant or sensitive to treatment.

While the method of the invention is suitable for use on any patient having a cancer, preferably the patient is a human. The patient may be afflicted with any cancer, such as, for example, breast, colon, prostate, and squamous cell carcinoma, to list only a few. Preferably the cancer is one that is accessible for biopsy. In a more preferred embodiment, the cancer is a squamous cell carcinoma, most preferably, a squamous cell carcinoma of the head and neck.

The cancer treatment methods used herein may be any treatment method known in the art, such as, surgery, radiation therapy, chemotherapy, and any and all combinations thereof. Preferably the cancer treatment of the present invention is radiation therapy. More preferably, the treatment is concomitant radiation therapy and chemotherapy. The protocols for treatment comprising dosage and duration of treatment are known and established in the art.

Even another embodiment of the invention is directed to a method of making a molecular chip of the invention. The molecular chips of the invention are described above. Generally the method of making said chips comprises an initial step of determining treatment-resistant and treatment-sensitive markers using an analysis such as cDNA array analysis on at least one treatment-resistant control sample and at least one treatment-sensitive control sample. The markers may be any large group of genes that are differentially expressed in the treatment-sensitive and treatment-resistant control samples. Thus, the prognostic molecular markers have a treatment-sensitive expression intensity and a treatment-resistant expression intensity. The selection of genes for use as molecular markers is based on at least the following criteria: the ratio of treatment-sensitive expression intensity and treatment-resistant expression intensity is greater than about 3. If a ratio cannot be determined for the markers, then the absolute difference between treatment-sensitive expression intensity and treatment-resistant expression intensity is preferably greater than about 75. The expression intensities may be determined by any technique known in the art for simultaneously assaying expression patterns of a large number of genes. In a preferred embodiment, a cDNA array assay is utilized. As described above, apparatus for manufacturing a molecular chip are known in the art such as, for example, those discussed at the internet site of Dr. Leming Shi, web address www.gene-chips.com/ and references therein, and are included herein. Techniques for adhering molecules to chips are also known in the art and all such techniques are applicable for the present invention.

Still another embodiment of the invention is directed to a group of prognostic molecular marker genes, wherein the genes exhibit differential expression patterns in a treatment-resistant control sample and in a treatment-sensitive control sample. The genes of the group may be any genes however, to be selected for use as a molecular marker, the ratio of treatment-resistant expression intensity and treatment-sensitive expression intensity of the gene must be greater than about 3-fold. Preferably the treatment is radiation therapy. In addition, the group may comprise any number of genes.

The present invention is also directed to any and all methods for predicting the treatment response of a cancer. Generally the method comprises assaying a patient sample for the expression intensity of treatment-sensitive and treatment-resistant markers. Preferably the assay is a cDNA array hybridization on a molecular chip and the treatment is radiation therapy.

All references cited herein, including research articles, all U.S. and foreign patents and patent applications, are specifically and entirely incorporated by reference.

### EXAMPLES

The following examples are provided to illustrate the present invention. These examples are not intended to limit the scope of the claims of the present invention, and should not be so interpreted.

### Example 1

### RNA isolation from Biopsy and Surgical Samples.

Squamous cell carcinoma (SCC) biopsy and surgical tissues were obtained from patients undergoing surgical resection at the University of Arkansas for Medical Sciences (UAMS), with the protocol approved by the UAMS institutional review board. After surgical removal, samples were immediately snap-frozen in liquid nitrogen. Tumor tissues were homogenized in TRIzol reagent (Life Technologies, Inc., Rockville, Md.) with a bead-beater to extract total RNA (Elek, J., Park, K. H., and Narayanan, R. Microarray based expression profiling in prostate tumors. In Vivo 14: 173-182, 2000).

### Example 2

### Atlas™ cDNA Array Analysis

RNA, 30 μg, was treated with 10 units of DNase I (MessageClean Kit, GenHunter Corp., Nashville, Tenn.) for 30 minutes at 37° C. to digest any contaminating DNA. Five μg of total RNA from each sample were converted into 32p-labeled first-strand cDNA by reverse transcription using gene-specific primers, according to the manufacturer's specifications (Clontech Laboratories, Inc.). Probes were purified and hybridized to the filter array overnight at 68° C. Filters were washed and exposed to a Storage phosphor screen (Molecular Dynamics, Sunnyvale, Calif.,), and differences in the signals among samples were scanned by a PhosphorImager analyzer (Model 445 SI, Molecular Dynamics,) and analyzed by Atlas Imagem 1.5 software (Clontech).

For normalization, the most consistent results were obtained by choosing a global method. The filters hybridized with radiation-sensitive and radiation-resistant probes were exposed in a manner such that a similar signal intensity was produced from both filters. The filters containing 1,187 unique human cancer-related genes are available from Clontech.

Sixty genes out of 1,187 were determined to be prognostic genes for radiation response based on two criteria: 1) the ratio of gene expression between radiation-resistant and -sensitive samples was greater than 3-fold; and 2) the absolute difference was greater than 75, if the ratio was undefined during the analysis. Interestingly, these genes include some of the classically known radiation-responsible genes such as c-jun and XRCC1, but many genes listed in Table 1 and Table 2 have not been previously associated in the art with radiation response.

TABLE 1
The selected radiation-resistant genes fromthe Atlas human cancer 1.2 array analysis

| Gene code | Ave S3 | Ave R2 | R:S | Difference | Protein/gene |
|---|---|---|---|---|---|
| A01c | 393 | 1259 | 3.2 | 868 | c-jun proto-oncogene: transcription factor AP-1 |
| A06i | 36 | 144 | 4.0 | 108 | G1/S-specific cyclin D1 (CCND1); bcl-1 oncogene |
| A07b | 9 | 58 | 6.4 | 49 | Tight junction protein zonula occludens (ZO-1); |
| A09h | 13 | 99 | 7.6 | 86 | P126, (ST5) |
| B02m | 51 | 203 | 4.0 | 152 | Unit protein; protein kinase C inhibitor 1 (PKC11) |
| B04i | 18 | 61 | 3.4 | 43 | Phosphotyrosyl phosphotase activator |
| B12l | 142 | 626 | 4.4 | 484 | STAT-induced STAT inhibitor 3 |
| C12n | 33 | 118 | 3.6 | 85 | Checkpoint suppressor 1 |
| D02f | 28 | 117 | 4.2 | 89 | Colon carcinoma laminin-binding protein: NEM/1CHD4 |
| D06B | 46 | 199 | 4.3 | 153 | Nonhistone chromosomal protein HMG17 |
| D10k | 47 | 153 | 3.3 | 106 | Tumor necrosis factor receptor 2-related protein; |
| D11b | 105 | 450 | 4.3 | 345 | Bone proteoglycan 11 precursor (PGS2): decorin (DCN) |
| D14n | 8 | 125 | 15.6 | 117 | Semaphonin |
| E02f | 84 | 252 | 3.0 | 168 | Insulin-like growth factor-binding protein 3 precursor |
| E06n | 0 | 84 | Undefined^{a} | 84 | Procollagen 1α 2 subunit precursor (COL1A2) |
| E07i | 163 | 1364 | 8.4 | 1201 | Matrix metalloprotcinase 3 (MMP3): |
| E09h | 175 | 526 | 3.0 | 351 | Jagged2 (JAG2) |
| E10m | 7 | 158 | 22.6 | 151 | MHC class II HLA-DR-β |
| E13m | 291 | 929 | 3.2 | 638 | Immunoglobulin α 1 heavy chain constant region |
| F03m | 18 | 63 | 3.5 | 45 | HEM4S |
| F04m | 72 | 287 | 4.0 | 215 | Polyhomeolic 2 homolog (IIP112) |
| F09c | 57 | 273 | 4.8 | 216 | Uridline phosphorylase (UDR Pase; UP) |

^{a}The ratio cannot be determined

TABLE 2
The selected radiation-sensitive genes from the Atlas human cancer 1.2 array analysis

| Gene code | Ave S3 | Ave R2 | S:R | Difference | Protein/gene |
|---|---|---|---|---|---|
| A07i | 111 | 36 | 3.1 | −75 | G1/S-specific cyclin D2 (CCND2) + KIAK0002 |
| A10f | 437 | 77 | 5.7 | −360 | Matrix metalloproteinase II (MMP11); stromelysin 3 |
| A10k | 109 | 34 | 3.2 | −75 | Cyclin-dependent kinase regulatory subunit 1 (CKS1) |
| A14c | 197 | 61 | 3.2 | −136 | ets-related protein tel: ets translocation variant 6 (ETV6) |
| B01C | 107 | 32 | 3.3 | −75 | Ribosomal protein S6 kinase II alpha 3 (S6KII-alpha 3): |
| B03B | 255 | 79 | 3.2 | −176 | α-2-macroglobulin receptor-associated protein precursor (alpha-2-MRAP; |
| B10C | 98 | 32 | 3.1 | −66 | c-jun N-terminal kinase 2 (JNK2); JNK55 |
| C03A | 107 | 30 | 3.6 | −77 | Tumor necrosis factor receptor (TNFR) + tumor necrosis factor receptor 2 |
| C04b | 398 | 130 | 3.1 | −268 | Tumor necrosis factor type 1 receptor associated protein (TRAP1) |
| C08g | 142 | 32 | 4.4 | −110 | mutL protein homolog; DNA mismatch repair protein MLH1: COCA2 |
| D06m | 168 | 39 | 4.3 | −129 | Cytosolic superoxide dismulase I (SOD1) |
| D08d | 189 | 36 | 5.3 | −153 | Thrombospondin 2 precursor (THBS2: TSP2) |
| E05i | 1608 | 335 | 4.8 | −1273 | Matrix metalloproteinase 1 (MMP1); interstitial collagenase precursor (CLG) |
| F05h | 344 | 48 | 7.2 | −296 | Interferon-regulated resistance GTP-binding protein MXA (IF1-78K): |
| F06d | 285 | 86 | 3.3 | −199 | _{L}-Lactate dehydrugenase II subunit (LD11B) |
| F09f | 553 | 58 | 9.5 | −495 | Type I cytoskeletal 19 keratin: cytokeratin 19 (K19; CK19) |
| F12f | 2357 | 407 | 5.8 | −1950 | Type II cytoskeletal 2 epidermal keratin (KRT2E); cytokeratin 2E (K2E; CK2E) |
| A08b | 85 | 0 | Undefined^{a} | −85 | Mothers against dpp homolog 4 (SMAD4); MADR4; pancratic carcinoma gene 4 |
| A12c | 104 | 0 | Undefined | −104 | fos-related antigen 2 (FRA2) |
| A12j | 93 | 0 | Undefined | −93 | cdc2-related protein kinase PISSLRE |
| B01c | 113 | 0 | Undefined | −113 | Dual-specificity mitogen-activated protein kinase kinase 1 |
| B06a | 127 | 0 | Undefined | −127 | Insulin-like growth factor binding protein 5 precursor (IGF-binding protein 5;) |
| C01h | 99 | 0 | Undefined | −99 | DNA-repair protein XRCCI |
| C04g | 85 | 0 | Undefined | −85 | DNA excision repair protein ERCCI |
| C09n | 247 | 0 | Undefined | −247 | Acid finger protein |
| D02c | 118 | 0 | Undefined | −118 | Lymphocyte antigen |
| D05h | 76 | 0 | Undefined | −76 | Platelet-derived growth factor receptor beta subunit (PDGFRB); CD140B antigen |
| D06e | 84 | 0 | Undefined | −84 | Mesothelin precursor, CAK1 antigen |
| D07h | 88 | 0 | Undefined | −88 | CDW40 antigen; CD40L receptor precursor, |
| E03m | 100 | 0 | Undefined | −100 | HLA-DPBI precursor; IILA class II histocompability antigen SB beta chain |
| E13h | 85 | 0 | Undefined | −85 | Wnt-5A |
| E14j | 96 | 0 | Undefined | −96 | Matrix metalloproteinase 17 (MMP17); |
| F03g | 272 | 0 | Undefined | −272 | Type II cytoskeletal 8 keratin (KRT8); cyrokeratin 8 (K8; CK8) |
| F05f | 159 | 0 | Undefined | −159 | Type I cytoskeletal 13 keratin; |
| F10m | 88 | 0 | Undefined | −88 | IFN-α-induced 11.5-kDa protein |
| F07a | 86 | 0 | Undefined | −86 | Laminin β-1 subunit precursor (laminin B1: LAMB1) |
| F13f | 167 | 0 | Undefined | −167 | Type II cytokeletal 4 keratin (KRT4): cytokeratin 4 (K4; CK4) |

^{a}The ratio cannot be determined.

### Example 3

### Cluster Analysis.

If the ratio of gene expression between the radiation sensitive and resistant tumor tissues changed more than 3-fold, and/or the absolute difference was more than 75, then the gene was selected for cluster analysis after the transformation of raw observation (adjusted intensity) using the equation: y=log 10 (x+1), where x equals the adjusted intensity from array analysis, and y is the transformed adjusted-intensity. The cluster analysis was performed using weighted pair-group average and Euclidean distance available in the software STATISTICA (StatSoft, Tulsa, Okla.). A tree diagram was generated by the software.

The cluster analysis enabled organization of the 60 selected molecular marker genes into 3 categories as follows: 1) a group with undetectable gene expression in the radiation-resistant sample and relatively high level of gene expression in the radiation-sensitive sample (with an absolute expression difference of 75); 2) a group with genes over-expressed in radiation-resistant samples; and 3) a group with genes over-expressed in radiation sensitive tissues. The last two groups had gene expression ratios greater than 3-fold.

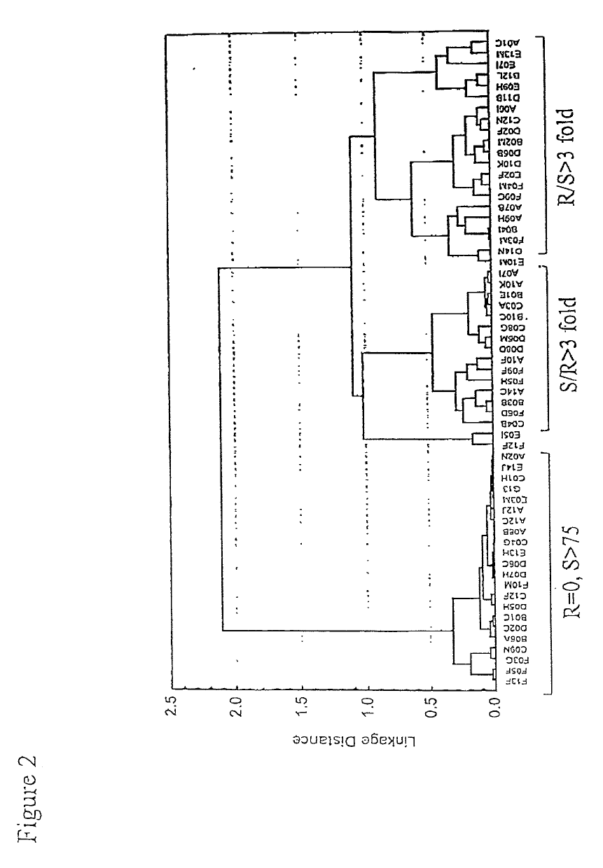

The three groups are shown in FIG. 2. The left group was composed of genes with a higher level of gene expression in radiation-resistant tissue, the middle group was formed from genes with greater than 3-fold expression in the sensitive tissue, and the right group represents genes with high levels of expression in radiation-sensitive tumors but not detectable expression levels in radiation resistant-tissue.

### Example 4

### Prediction of Radiation Response of Test Samples by Cluster Analysis Using 60 Selected Genes.

The present example determined whether any of the 60 molecular markers genes selected from the gene-expression profiles of radiation-sensitive and radiation-resistant groups could predict the radiation response of a tumor sample. The gene-expression profile was determined from two different samples using the same array filter and method in a single blind approach. These two samples were known to be radiation-sensitive based on their clinical response.

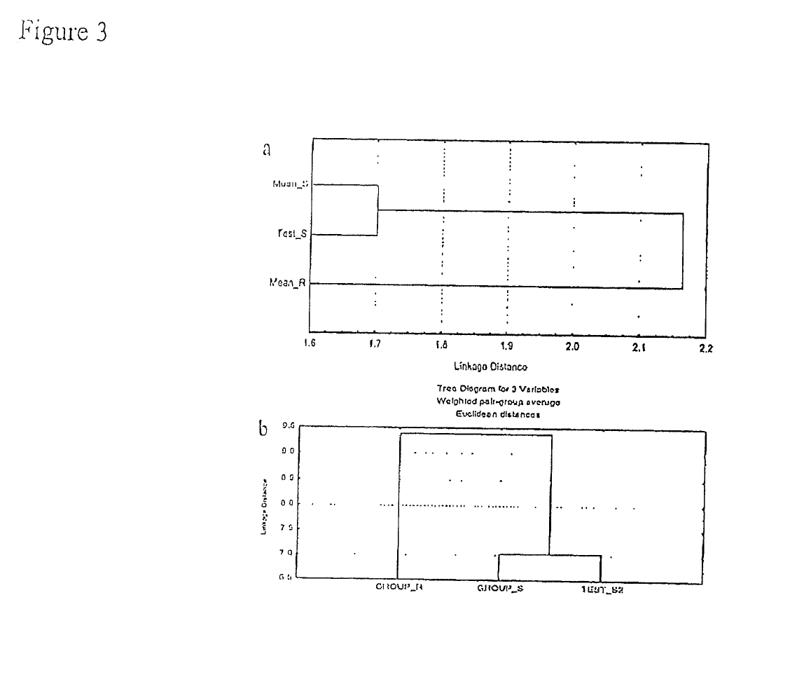

Following the gene probe hybridization and scanning analysis, the adjusted-intensity of the 60 genes was determined and transformed as described for the method of the invention. The transformed intensity was clustered with the known sensitive and resistant groups described above in Example 3 using the 60 selected-gene expression profiles to determine the initial cluster group. As expected, the first radiation-sensitive sample had a much shorter cluster distance than the radiation-resistant group and was clustered with the radiation-sensitive group first (shown in FIG. 3A). Similarly, the second radiation-sensitive tumor sample that was tested also clustered with the sensitive group first (shown in FIG. 3B).

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein, but rather that the claims be construed as encompassing all the features of patentable novelty which reside in the present invention, including all features which would be treated as equivalents thereof by those skilled in the art to which this invention pertains.

All references cited in the present application, including journal articles, U.S. and foreign patents and patent applications, are herein incorporated by reference. The following publication by the inventors is also incorporated herein by reference: Hanna E. et al., Cancer Research 61, 2376-2380, Mar. 15, 2001.

We claim:

1. A molecular chip comprising an array of nucleic acid sequences specific for a group of molecular marker genes, wherein the genes exhibit a treatment-resistant expression intensity and a treatment-sensitive expression intensity, and wherein the ratio of treatment-resistant expression intensity and treatment-sensitive expression intensity is greater than about 3-fold.

2. The chip of claim 1 wherein the treatment is radiation therapy.

3. The chip of claim 1 wherein the cancer is a squamous cell carcinoma of the head and neck.

4. The chip of claim 1 wherein the patient is a human.

5. The chip of claim 1 wherein the molecular chip comprises a cDNA array.

6. The chip of claim 1 wherein the patient sample is a biopsy.

7. A method for predicting treatment response of a cancer in a patient, the method comprising the steps of:
a) contacting a portion of a sample from a cancer of a patient together with a molecular chip to obtain an expression intensity pattern of the sample;
b) transforming the expression intensity pattern of the patient sample with the equation y=log 10 (x+1), wherein x equals the expression intensity and y equals the transformed expression intensity.

8. The method of claim 7 further comprising the step of:
c) performing cluster analysis on the transformed expression intensity, wherein the cluster analysis comprises weighted pair-group average and Euclidian distance.

9. The method of claim 7 wherein the chip comprises an array of nucleic acid sequences specific for a group of molecular marker genes, wherein the genes exhibit a treatment-resistant expression intensity and a treatment-sensitive expression intensity, and wherein the ratio of treatment-resistant expression intensity and treatment-sensitive expression intensity is greater than about 3-fold.

10. The method of claim 7 wherein the treatment is radiation therapy.

11. The method of claim 7 wherein the cancer is a squamous cell carcinoma of the head and neck.

12. The method of claim 7 wherein the patient is a human.

13. The method of claim 7 wherein the molecular chip comprises a cDNA array.

14. The method of claim 7 wherein the patient sample is a biopsy.

15. A group of prognostic molecular marker genes, wherein the genes exhibit a treatment-resistant expression intensity and a treatment-sensitive expression intensity, and wherein the ratio of treatment-resistant expression intensity and treatment-sensitive expression intensity is greater than about 3-fold.

16. The group of claim 15 wherein the treatment is radiation therapy.
